(19) United States
(12) Patent Application Publication
(10) Pub. No.: US 2012/0079612 A1
(43) Pub. Date: Mar. 29, 2012

(54) Dry eye animal model

(75) Inventors: Jennifer Anne Sivak-Callcott, Morgantown, WV (US); Charles L. Rosen, Morgantown, WV (US); Jason D. Huber, Morgantown, WV (US)

(73) Assignee: West Virginia University

(21) Appl. No.: 13/200,221

(22) Filed: Sep. 21, 2011

Related U.S. Application Data
(60) Provisional application No. 61/403,977, filed on Sep. 24, 2010.

(51) Int. Cl. A01K 67/027

(52) U.S. Cl. 800/9

(57) ABSTRACT

A first embodiment is a dry eyed animal model method by peri or post-menopausal estrogen-treated rats have decreased tear production wherein the menopausal rat may be produced by ovariectomy Chronic estrogen exposure can decrease tear production in rats receiving a nine month course of estrogen versus placebo treatment after ovariectomy. The aged, chronically estrogen-treated female rats can provide a suitable model for the study of KCS and its treatment.

### CROSS-REFERENCE TO RELATED APPLICATIONS

This Application claims priority to provisional application Ser. No. 61/403,977 filed on Sep. 24, 2010.

### REFERENCE TO SEQUENCE LISTING, A TABLE, OR A COMPUTER PROGRAM LISTING COMPACT DISC APPENDIX

Not Applicable

### BRIEF DESCRIPTION OF THE SEVERAL VIEWS OF THE DRAWINGS

FIG. 1 is a graph of the three experimental groups.

FIG. 2 is a graph of tear breakup time for the three groups.

FIG. 3 is a graph of tear volume for the three groups.

FIG. 4 is a graph of E2 levels in rats treated with E2 and non-treated, Intact rats.

FIG. 5 is a graph of Tear Volume and Tear Break-up Time for post-menopausal E2 treated rats.

### DETAILED DESCRIPTION OF THE INVENTION

Dry eye syndrome or keratoconjunctivitis sicca (KCS) affects nearly 25 million people in the U.S. and is a leading cause of patient visits to both ophthalmologists and optometrists.¹ A normal tear film is essential for the health of the eye. Abnormal or insufficient tear film causes ocular surface damage, including corneal changes that can lead to temporary or permanent visual impairment. The normal tear film consists of three layers: a mucin layer produced by conjunctival epithelium, an aqueous layer secreted by the lacrimal glands, and a lipid layer made by the eyelid Meibomian and Zeiss glands. Dry eye syndrome is most commonly caused by an inadequate production of the aqueous layer by the lacrimal glands. Management of KCS can be costly and includes topical lubricants, immunosuppressive agents, punctual occlusion, and surgery.

The tear film is affected by hormone replacement therapy. A 2001 report from the Women's Health Study demonstrated a nearly 30% increase of KCS in women taking hormone replacement therapy (HRT), and women with prior hysterectomy taking estrogen alone experienced an almost 70% increase in KCS compared to non-treated women of the same age.¹ Moreover, there are two reports of estrogen therapy inducing Sjogren's syndrome, an autoimmune disease that targets salivary glands, including the lacrimal gland, leading to KCS.² These studies show that estrogen is linked to decreased tear production, namely the aqueous component, which is produced by serous cells of the lacrimal gland.

A first embodiment is a dry eyed animal model method using peri or post-menopausal, estrogen-treated rats that have decreased tear production. Chronic estrogen exposure can decrease tear production in peri or post-menopausal rats receiving supplementation with 17 β-estradiol (E2) sufficient to produce physiological levels of serum estradiol such as a three to nine month course of E2 compared to rats receiving placebo treatment. The aged, chronically estrogen-treated female rats can provide a suitable model for the study of KCS and its treatment. Additionally, peri or post-menopausal rats can be produced with decreased tear production by ovariectomizing rats prior to menopause and treating with estrogen.

Female Sprague-Dawley rats have a life span of 24-27 months. The females are sexually mature between 1-3 months and maintain the ability to breed until menopause, which typically starts around 14 months. Females are typically retired from breeding around 9 months due to declining estrogen levels during the peri-menopausal period between 9 and 14 months. Peri-menopausal rats are about 9 to about 14 months in age while the post-menopausal rats are about 14 months or older.

### EXAMPLE 1

### Pre-Menopausal Treatment

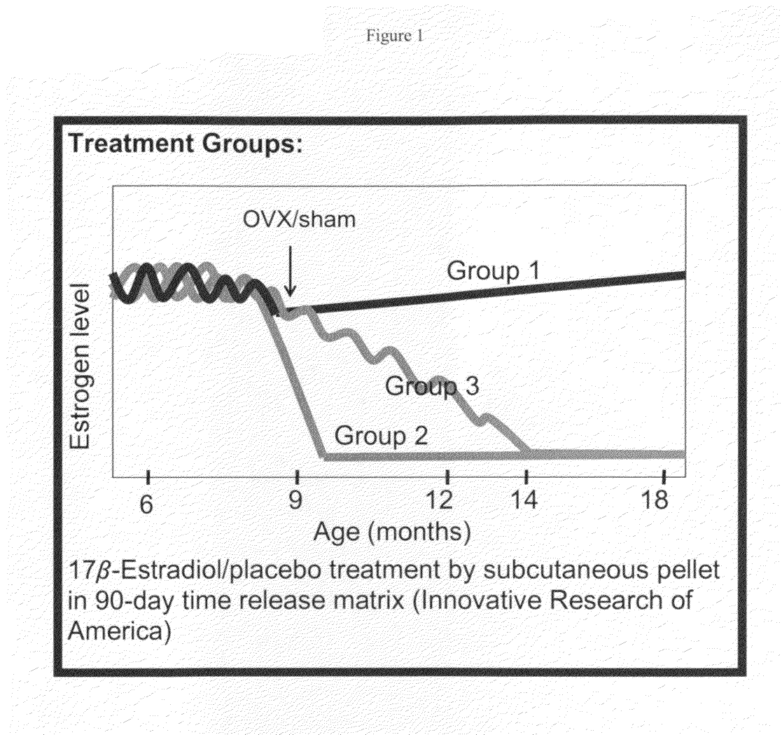

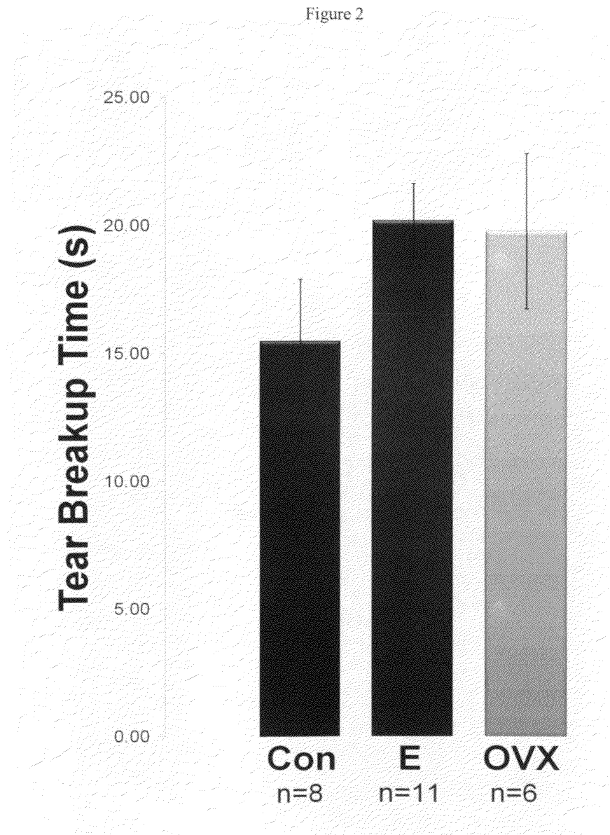

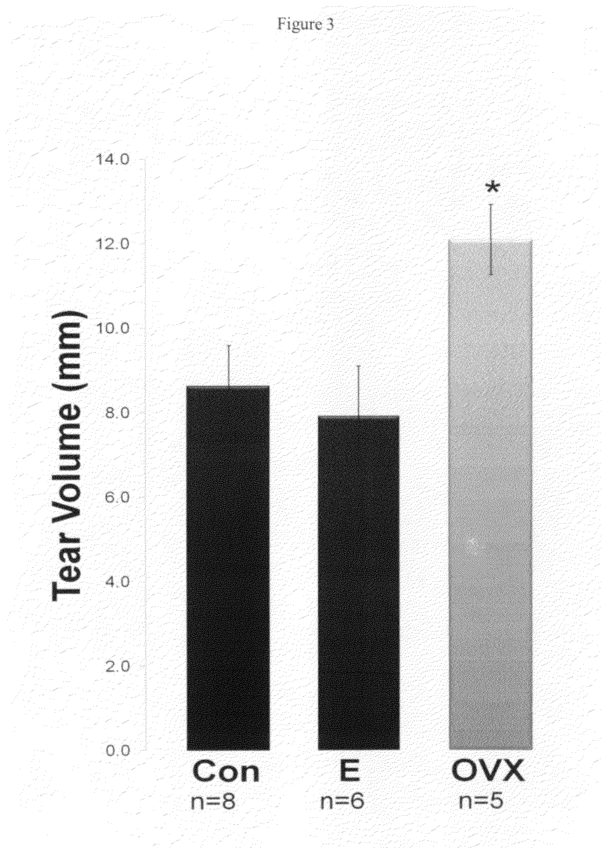

Female rats of 9 months of age were ovariectomized and immediately supplemented with 1.5 mg of 17β-estradiol in three, separate 90-day time release pellets until 18 months. Three experimental groups were used with ovaries excised from rats in treatment Groups 1 (Intact) and 2 (OVX), but left intact in rats in Group 3 (E2) (FIG. 1). Group 1 underwent a sham ovariectomy and was implanted with placebo pellet, Group 2 underwent an ovariectomy and was implanted with placebo pellet, and Group 3 underwent an ovariectomy and was implanted with E2 pellet. The three groups had tear breakup time and tear volume tested. Tear breakup time was tested with 2 drops of Fluress instilled into 1 eye with a micropipette calibrated to 5 μl. The eyelids were manually closed 3 times. The time lapse between the last blink and the first randomly distributed dry spot on the cornea using a portable slit lamp was recorded. The process was repeated 3 times for each subject and the results recorded (FIG. 2). Tear volume was tested with a modified Schirmer's test. Schirmer's test strips were cut to 1.67 mm (+/−0.01 mm) width and folded at the 5 mm mark. Rats received 0.5 uL Fluress onto the eye, then the eye was manually blinked 3 times, and the modified Schirmer's strip was placed in the upper fornix for 5 minutes for assessment of tear volume and the volume was recorded (FIG. 3). The results in FIGS. 2 and 3 show reduced tear volume and shorter tear break up time with Group 3 when compared to the control groups 1 and 2.

### EXAMPLE 2

### Post-Menopausal Treatment

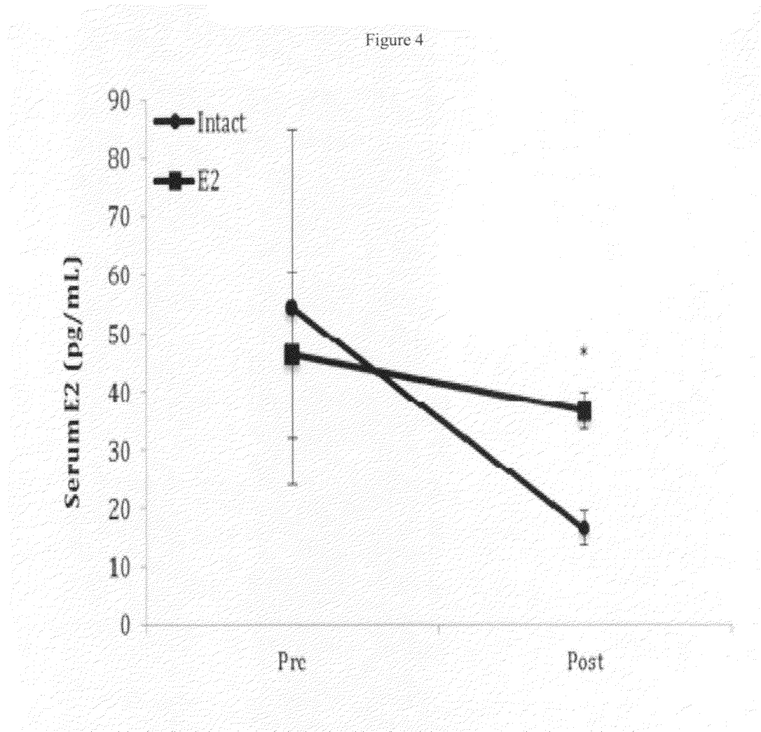

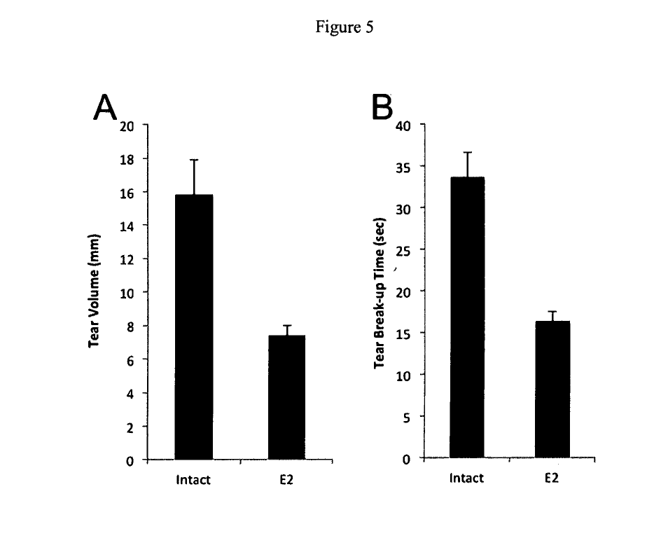

Female Sprague-Dawley rats, age 15 months, were subcutaneously implanted with placebo pellets and E2 rats received pellets containing 1.5 mg of 17β-estradiol in a 90-day time-release matrix. Both groups received treatment until they reached 18 months of age. Serum E2 levels were measured from blood collected prior to randomization to treatment groups and again at time of euthanasia. At 18 months, two distinct treatment groups could be differentiated by serum E2 levels with E2 rats having significantly elevated serum E2 levels compared to Intact rats (FIG. 4). Serum E2 levels of rats prior to randomization at 15 months of age (Pre) and two weeks before gland excision, at 18 months of age (Post). Rats treated with pellets containing 17β-estradiol had significantly higher serum E2 levels at 18 months compared to rats administered placebo pellets. Also, Tear Volume Assessment and Tear Break-up Time both have been examined in the rats. The aged rats treated with E2 had reduced tear volume and shorter Tear Break-up Time. Aged rats receiving E2 had significantly less tear volume compared to Intact rats (7.4±0.58 mm and 15.8±2.1 mm, respectively; p<0.001). TBUT was also altered by estrogen treatment with E2 rats having a significant reduction in time to first tear disruption compared to Intact rats (16.4±1.1 sec and 33.7±1.9 sec, respectively; p<0.001) (FIG. 5). Tear volume was measured in 18 month old female rats during 3 months of E2 or placebo supplementation. Intact rats had significantly higher tear volume as measured by the modified Schirmer's I test compared to E2 rats. (B) Tear break up time was also measured in rats receiving placebo or E2 treatment using fluorescein sodium (0.25%). Time to first break up of tear film, as observed with slit lamp microscopy, was significantly higher in Intact rats compared to E2 rats. #p<0.001 compared to E2 rats using one-way ANOVA with Tukey's post hoc analysis.

Ovariectomy.

Rats can be anesthetized with isoflurane (4% induction; 2% maintenance). Once anesthetized, the rats can be placed on a surgical platform and the lateral abdominal regions are prepped for surgery using aseptic technique. A 10 mm dorsal/ventral incision into the lateral abdomen between the rib cage and hind limb can be made through the skin. The underlying abdominal muscle can be separated with iridectomy scissors to expose the ovarian fat pad. The ovaries can be excised, the muscle layer sutured, and the skin incision closed using 5-0 absorbable sutures. Topical antibiotic can be applied.

Estrogen Implementation.

The rats can be placed in the prone position on a surgical platform and the neck prepped using aseptic technique. A small incision can be placed at the base of the neck and a 3 month sustained release estrogen pellet containing 1.5 mg of 17β-estradiol can be inserted into a bluntly dissected cavity between the skin and muscle in the lateral aspect of the neck. The incision can be closed with 5-0 absorbable suture. The procedure can be repeated every three months.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

### REFERENCES

1. Schaumberg D A, Buring J E, Sullivan D A, Dana M R. Hormone replacement therapy and dry eye syndrome. JAMA 2001;286:2114-19.
2. Nagler R M, Pollack S. Sjogren's syndrome induced by strogen therapy. Seminars in Arthritis and Rheumatism 2000;30:209-14.

What is claimed:

1. A method comprising producing a rat with reduced tear production and shorter tear break-up time wherein the rat is peri or post-menopausal and treated with a sufficient 17β-estradiol to produce physiological levels of serum estradiol in the rats.

2. The method of claim 1 wherein the peri or post-menopausal rat is a premenopausal rat ovariectomized to create a peri or post-menopausal rat.

3. The method of claim 1 wherein 17β-estradiol treatment is 1.5 mg of estradiol in a 90-day time release pellet.

4. The method of claim 1 wherein the rats are Sprague-Dawley rats.
